(19) United States
(12) Patent Application Publication
(10) Pub. No.: US 2014/0004540 A1
(43) Pub. Date: Jan. 2, 2014

(54) METHODS AND KITS FOR PREDICTING THE RISK OF HAVING A CARDIOVASCULAR EVENT IN A SUBJECT

(71) Applicants: Ziad Mallat, Paris (FR); Alain Tedgui, Paris (FR); Tabassome Simon, Paris (FR); Nicolas Danchin, Paris (FR)

(72) Inventors: Ziad Mallat, Paris (FR); Alain Tedgui, Paris (FR); Tabassome Simon, Paris (FR); Nicolas Danchin, Paris (FR)

(73) Assignee: UNIVERSITE PARIS DESCARTES, Paris Cedex 06 (FR)

(21) Appl. No.: 13/979,400

(22) PCT Filed: Jan. 13, 2012
(86) PCT No.: PCT/EP2012/050474

§ 371 Date: Sep. 17, 2013

(30) Foreign Application Priority Data
Jan. 13, 2011 (EP) 11305032.2
Mar. 21, 2011 (EP) 11305318.5

(51) Int. Cl. G01N 33/68

(52) U.S. Cl. CPC G01N 33/6869
USPC 435/7.92

(57) ABSTRACT

The present invention relates to in vitro methods and kits for predicting the risk of having a cardiovascular event in a subject. More particularly, the invention relates to an in vitro method for predicting the risk of a cardiovascular event in a subject, said method comprising the step of measuring the level of IL-17 in a blood sample obtained from said subject.

### FIELD OF THE INVENTION

The present invention relates to in vitro methods and kits for predicting the risk of having a cardiovascular event in a subject.

### BACKGROUND OF THE INVENTION

Atherosclerosis is a complex disease of the arterial wall initiated in response to a variety of pro-atherogenic stimuli, among which modified lipids play an important role. The latter induce innate and adaptive immune responses with both deleterious and protective components. A breakdown in this balance leads to uncontrolled disease progression and precipitates severe complications.

Our current understanding is that the deleterious component of the adaptive immune response is driven by T helper type 1 (Th1)-related mediators, mainly interferon (IFN)-γ, which play central roles in promoting vascular inflammation, artery wall remodelling and plaque progression. The protective component is still poorly understood. However, several studies have shown that 3 cytokines play critical counter-regulatory roles in atherosclerosis. Interleukin (IL)-5, a Th2-related cytokine, links adaptive and natural immunity to epitopes of oxidized (ox) low-density lipoproteins (LDL) and stimulates the expansion of atheroprotective natural IgM antibodies specific for oxLDL. IL-10 and transforming growth factor (TGF)-β, two major cytokines linked to the family of regulatory T (Treg) cells, play important non-redundant atheroprotective roles through their anti-inflammatory, immunosuppressive and vasculo-protective properties.

Recently, a new lineage of CD4+ T cells, called Th17, has been identified and characterized. Th17 produce IL-17A (hereafter referred to as IL-17), IL-17F, IL-21 and IL-22. Specific transcription factors such as retinoic acid-related orphan receptors (ROR)γt and ROR-α, as well as signal transducer and activator of transcription (STAT)3, are involved in the development of Th17, with the contribution of mediators for lineage differentiation (combination of IL-6 with TGF-β or IL-1β) and maintenance (IL-23). The discovery of Th17 lineage has revived a great interest in the potential roles of IL-17 in health and disease.

Several studies have shown a non-redundant role for IL-17 in the clearance of specific pathogens that are not adequately controlled by Th1 or Th2 immunity, particularly extracellular bacteria and fungi. Beyond this role in host defence, several researchers have implicated Th17 cells and cytokines in the pathophysiology of immune-mediated diseases, such as rheumatoid arthritis, psoriasis, colitis or asthma, even though a definite proof of pathogenic role is still lacking in humans.

On the basis of these data, several investigators hypothesized a deleterious role for IL-17 in atherosclerosis. Their results showed that IL-17 was expressed in human coronary and carotid atherosclerotic lesions, and reported enhanced production of pro-inflammatory mediators by vascular smooth muscle cells in response to IL-17, with or without help from IFN-γ (Eid RE, Rao DA, Zhou J, et al. Interleukin-17 and interferon-gamma are produced concomitantly by human coronary artery-infiltrating t cells and act synergistically on vascular smooth muscle cells. Circulation. 2009;119:1424-1432.; Patel D N, King C A, Bailey S R, et al. Interleukin-17 stimulates c-reactive protein expression in hepatocytes and smooth muscle cells via p38 mapk and erk1/2-dependent nf-kappab and c/ebpbeta activation. J Biol Chem. 2007;282:27229-27238.). Other experimental data showed elevated expression of IL-17 at the early stages of lesion development compared to nonatherosclerotic animals (Xie J J, Wang J, Tang T T, et al. The th17/treg functional imbalance during atherogenesis in apoe(−/−) mice. Cytokine. 2010;49:185-193.), and reported reduction of atherosclerosis after inhibition of IL-17 signalling (van Es T, van Puijvelde G H, Ramos O H, et al. Attenuated atherosclerosis upon il-17r signaling disruption in ldlr deficient mice. Biochem Biophys Res Commun. 2009;388:261-265. ; Erbel C, Chen L, Bea F, et al. Inhibition of il-17a attenuates atherosclerotic lesion development in apoe-deficient mice. J Immunol. 2009;183:8167-8175; Smith E, Prasad K M, Butcher M, et al. Blockade of interleukin-17a results in reduced atherosclerosis in apolipoprotein e-deficient mice. Circulation. 2010;121:1746-1755.).

However, another set of data suggests a regulatory role for IL-17 in atherosclerosis. We have recently shown that mice with T cells deficient for suppressor of cytokine signalling (SOCS)3 display elevated levels of IL-17, associated with reduced atherosclerotic lesion size (Taleb S, Romain M, Ramkhelawon B, et al. Loss of socs3 expression in t cells reveals a regulatory role for interleukin-17 in atherosclerosis. J Exp Med. 2009;206:2067-2077.). Systemic blockade of IL-17 signalling abrogated atheroprotection and promoted vascular inflammation (Taleb S, Romain M, Ramkhelawon B, et al. Loss of socs3 expression in t cells reveals a regulatory role for interleukin-17 in atherosclerosis. J Exp Med. 2009;206:2067-2077.). The results were related to the role of IL-17 in the regulation of endothelial vascular cell adhesion molecule (VCAM)-1 and to the control of both Th1 and Th2 responses. In addition, we found that elevated expression of IL-17 in human carotid lesions was associated with signs of plaque stability (Taleb S, Romain M, Ramkhelawon B, et al. Loss of socs3 expression in t cells reveals a regulatory role for interleukin-17 in atherosclerosis. J Exp Med. 2009;206:2067-2077.).

Overall, the relevance of IL-17 to human atherosclerosis remains poorly defined, and more importantly, its relevance to cardiovascular outcomes remains unexplored. This is an important issue given the current clinical testing of inhibitors of IL-17 signalling in immune-mediated diseases associated with a high cardiovascular risk. Therefore, the aim of the present study was to evaluate the relationship between circulating IL-17 levels and cardiovascular events, a composite of all-cause death and non-fatal myocardial infarction (MI), in subjects who suffered from acute MI. In addition, since IL-17 has been shown to regulate VCAM-1 expression in mice 23, we examined the role of IL-17 in mononuclear cell adhesion to endothelial cells and explored the relationship between IL-17, VCAM-1 and cardiovascular events.

### SUMMARY OF THE INVENTION

The present invention relates to in vitro methods and kits for predicting the risk of having a cardiovascular event in a subject. More particularly, the invention relates to an in vitro method for predicting the risk of a cardiovascular event in a subject, said method comprising the step of measuring the level of IL-17 in a blood sample obtained from said subject.

### DETAILED DESCRIPTION OF THE INVENTION

The Inventors' objectives were to assess the relationship between interleukin (IL)-17 levels and cardiovascular outcomes in subjects with acute myocardial infarction. Therefore the inventors assessed the relationship between serum levels of IL-17 and the risk of death and recurrent MI during the first year of follow-up in 981 subjects enrolled in the French registry of Acute ST elevation or non-ST-elevation Myocardial Infarction. Serum levels of IL-17 were associated with the risk of all-cause death and recurrent MI at one year, with levels of IL-17 below the median indicative of a worse outcome. The impact of IL-17 remained significant after adjustment for known cardiovascular risk factors, CRP, and treatments including statins: Hazard ratio (HR)=1.52 (1.06-2.20); P=0.02. IL-17 inhibited mononuclear cell adhesion to endothelium and reduced endothelial VCAM-1 expression. Subjects with low (below the median) IL-17 levels and high (above the median) soluble VCAM-1 (sVCAM-1) levels were at particularly increased risk of death and MI: adjusted HR=2.56 (1.31-5.04) compared with the high IL-17/low sVCAM-1 group (P=0.006). In conclusion, low serum levels of IL-17 are associated with a higher risk of major cardiovascular events in subjects with acute MI. These results suggest a protective regulatory role for IL17 in coronary artery disease and raise possible concern about the use of inhibitors of IL-17 pathway in clinical settings associated with a high cardiovascular risk.

### Methods of the Invention:

A first object of the invention relates to an in vitro method for predicting the risk of a cardiovascular event in a subject, said method comprising the step of measuring the level of IL-17 in a blood sample obtained from said subject.

According to the invention, the term “IL-17” has its general meaning in the art and refers to the interleukin-17A protein.

According to the invention, “cardiovascular event” is used interchangeably herein with the term “cardiac event”, “acute arteriovascular event”, or “arteriovascular event” and refers to sudden cardiac death, acute coronary syndromes such as, but not limited to, plaque rupture, myocardial infarction, unstable angina, as well as non-cardiac acute arteriovascular events such as blood clots of the leg, aneurysms or aneurysm progression, stroke and other arteriovascular ischemic events where arteriovascular blood flow and oxygenation is interrupted.

A “subject” in the context of the present invention is preferably a human, typically a Caucasian. A subject can be male or female. A subject can be one who has been previously diagnosed or identified as having arteriovascular disease or an arteriovascular event, and optionally has already undergone, or is undergoing, a therapeutic intervention for the arteriovascular disease or arteriovascular event. Alternatively, a subject can also be one who has not been previously diagnosed as having arteriovascular disease. For example, a subject can be one who exhibits one or more risk factors for arteriovascular disease, or a subject who does not exhibit arteriovascular risk factors, or a subject who is asymptomatic for arteriovascular disease or arteriovascular events. A subject can also be one who is suffering from or at risk of developing arteriovascular disease or an arteriovascular event. Typically, the subject has been diagnosed as presenting one of the following coronary disorders:
asymptomatic coronary artery coronary diseases with silent ischemia or without ischemia;
chronic ischemic disorders without myocardial necrosis, such as stable or effort angina pectoris;
acute ischemic disorders without myocardial necrosis, such as unstable angina pectoris;
ischemic disorders with myocardial necrosis, such as ST segment elevation myocardial infarction or non-ST segment elevation myocardial infarction.

As used herein “blood sample” includes whole blood, plasma, serum, circulating cells, constituents, or any derivative of blood.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Risk” in the context of the present invention, relates to the probability that an event will occur over a specific time period, as in the conversion to arteriovascular events, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no- conversion.

In one embodiment, the method of the invention further may comprise a step of comparing the level of IL-17 measured in the blood sample obtained from the subject with a reference value.

Accordingly, the present invention relates to an in vitro method for predicting the risk of a cardiovascular event in a subject, said method comprising the step of i) measuring the level of IL-17 in a blood sample obtained from said subject ii) comparing the level measured at step ii) with a reference value wherein a difference between said IL-17 level and said reference value is indicative of a risk of having a cardiovascular event.

In another embodiment, the method of the invention further comprises a step of measuring the level of sVCAM in the blood sample obtained from the subject.

The term “sVCAM-1” (soluble vascular cell adhesion molecule-1, also known as CD106) has its general meaning in the art and refers to soluble form of the sVCAM-1 protein.

More particularly, an aspect of the invention thus relates to a method for predicting the risk of a cardiovascular event in a subject, said method comprising the step of measuring the level of IL-17 and sVCAM-1 in a blood sample obtained from said subject, combining said measurements, wherein the combined value of IL-17 and sVCAM-1 being indicative of the risk of having a cardiovascular event.

In one embodiment of the invention, the combined value of IL-17 and sVCAM-1 levels is compared to a reference value.

In one embodiment, the reference values may be index values or may be derived from one ore more risk prediction algorithms or computed indices for cardiovascular event. A reference value can be relative to a number or value derived from population studies, including without limitation, such subjects having similar body mass index, total cholesterol levels, LDL/HDL levels, systolic or diastolic blood pressure, subjects of the same or similar age range, subjects in the same or similar ethnic group, subjects having family histories of atherosclerosis, atherothrombosis, or CAD, PAD, or CVD, or relative to the starting sample of a subject undergoing treatment for an arteriovascular disease, such as atherosclerosis, atherothrombosis, CAD, PAD, or CVD.

Such reference values can be derived from statistical analyses and/or risk prediction data of populations obtained from mathematical algorithms and computed indices of arteriovascular disease, such as but not limited to, algorithms reported in the Framingham Study, NCEP/ATP III, among others. Cardiovascular Risk Factor reference value can also be constructed and used using algorithms and other methods of statistical and structural classification.

In one embodiment of the present invention, the reference value is derived from the level of IL-17 (or the combined value of IL-17 and sVCAM-1 levels) in a control sample derived from one or more subjects who are substantially healthy as defined here above. Such subjects who are substantially healthy lack traditional risk factors for a cardiovascular disease: for example, those subjects have a serum cholesterol level less than 200 mg/dl, systolic blood pressure less than or equal to 120 mm Hg, diastolic blood pressure less than or equal to 80 mm Hg, non-current smoker, no history of diagnosed diabetes, no previously diagnosed acute coronary syndrome or hypertension, among other aforementioned other risk factors, or can be verified by another invasive or non-invasive diagnostic test of cardiovascular disease known in the art, such as but not limited to, electrocardiogram (ECG), carotid B-mode ultrasound (for intima-medial thickness measurement), electron beam computed tomography (EBCT), coronary calcium scoring, multi-slice high resolution computed tomography, nuclear magnetic resonance, stress exercise testing, angiography, intra- vascular ultrasound (IVUS), other contrast and/or radioisotopic imaging techniques, or other provocative testing techniques. In another embodiment, such subjects are monitored and/or periodically retested for a diagnostically relevant period of time (“longitudinal studies”) following such test to verify continued absence from cardiovascular disease or acute cardiovascular events (disease or event free survival). Such period of time may be one year, two years, two to five years, five years, five to ten years, ten years, or ten or more years from the initial testing date for determination of the reference value. Furthermore, retrospective measurement of IL-17 levels (or the combined value of IL-17 and sVCAM-1 levels) in properly banked historical subject samples may be used in establishing these reference values, thus shortening the study time required, presuming the subjects have been appropriately followed during the intervening period through the intended horizon of the product claim. Typically, the levels of IL-17 in a subject whois at risk for a cardiovascular event is deemed to be lower than the reference value obtained from the general population or from healthy subjects.

In another embodiment, a reference value can also be derived from IL-17 level (or the combined value of IL-17 and sVCAM-1 levels) in a sample derived from one or more subject who has been previously diagnosed or identified for a cardiovascular event.

In another embodiment, the method of the invention further comprises the steps of assessing other cardiovascular risk factor selected in the group of Framingham Risk Score (FRS), CRP, IgM ICof apoB 100 or IgM MDA-LDL, Lp-PLA2, sPLA2 activity and sPLA2 mass. “Cardiovascular Risk Factor” encompasses one or more biomarker whose level is changed in subjects having a cardiovascular disease or predisposed to developing a cardiovascular disease, or at risk of a cardiovascular event.

According to the invention, the measure of level of IL-17 can be performed by a variety of techniques. Typically, the methods may comprise contacting the sample with a binding partner capable of selectively interacting with IL-17 in the sample. In some aspects, the binding partners are antibodies, such as, for example, monoclonal antibodies or even aptamers as above described.

The aforementioned assays generally involve the binding of the partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The level of IL-17 may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

An exemplary biochemical test for identifying specific proteins employs a standardized test format, such as ELISA test, although the information provided herein may apply to the development of other biochemical or diagnostic tests and is not limited to the development of an ELISA test (see, e.g., Molecular Immunology: A Textbook, edited by Atassi et al. Marcel Dekker Inc., New York and Basel 1984, for a description of ELISA tests). It is understood that commercial assay enzyme-linked immunosorbant assay (ELISA) kits for various plasma constituents are available. Therefore ELISA method can be used, wherein the wells of a microtiter plate are coated with a set of antibodies which recognize IL-17. A sample containing or suspected of containing IL-17 is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Measuring the level of IL-17 (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e. g., retention time, for that compound and measured using standard techniques.

Alternatively, the separated compounds may be detected and measured by, for example, a mass spectrometer.

Typically, levels of immunoreactive IL-17 in a sample may be measured by an immunometric assay on the basis of a double-antibody “sandwich” technique, with a monoclonal antibody specific for IL-17 (Cayman Chemical Company, Ann Arbor, Mich.). Preferably, the antibody has no cross-reactivity with the other types of IL-17 such as IL-17B, IL-17C, IL-17D, IL-17E and IL-17F. According to said embodiment, said means for measuring IL-17 level are for example i) a IL-17 buffer, ii) a monoclonal antibody that interacts specifically with IL-17, iii) an enzyme-conjugated antibody specific for IL-17 and a reference value of IL-17.

According to the invention, the measure of level of sVCAM-1 can be performed similarly. Typically, the methods may comprise contacting the sample with a binding partner capable of selectively interacting with sVCAM-1 in the sample. In some aspects, the binding partners are antibodies, such as, for example, monoclonal antibodies or even aptamers as above described. Methods described above for the measure of the level of IL-17 may be similarly used.

In a particular embodiment, the method as described here above is particularly suitable for monitoring the effectiveness of a treatment for a cardiovascular disease. The efficacy of the treatment will be reflected by changes in the measurements of the IL-17 levels (or the combined values of IL-17 and sVCAM-1 levels). Typically, an efficient treatment will enable to get IL-17 levels that will increase compared to the levels of IL-17 measured before the treatment, suggesting that the risk for a cardiovascular event diminishes. In another embodiment of the invention, the method as described here above is for selecting a treatment regimen for a subject diagnosed with or at risk for a cardiovascular disease.

A further object of the invention relates to the use of circulating IL-17 as a biomarker of the risk of having a cardiovascular event in a subject.

In a particular embodiment, methods of the invention are also particularly useful for monitoring treatment with Th17 blockers. Indeed it has been showed that said treatment can be associated with major adverse cardiovascular events as reported in recent studies (Griffiths C E, Strober B E, van de Kerkhof P, Ho V, Fidelus-Gort R, Yeilding N, Guzzo C, Xia Y, Zhou B, Li S, Dooley L T, Goldstein N H, Menter A; ACCEPT Study Group.Comparison of ustekinumab and etanercept for moderate-to-severe psoriasis. N Engl J Med. 2010 Jan 14;362(2):118-28.; Krueger G G, Langley R G, Leonardi C, Yeilding N, Guzzo C, Wang Y, Dooley L T, Lebwohl M; CNTO 1275 Psoriasis Study Group. A human interleukin-12/23 monoclonal antibody for the treatment of psoriasis. N Engl J Med. 2007 Feb 8;356(6):580-92.; Reich K, Langley R G, Lebwohl M, Szapary P, Guzzo C, Yeilding N, Li S, Hsu M C, Griffiths C E. Cardiovascular safety of ustekinumab in subjects with moderate-to-severe psoriasis: Results of integrated analyses of data from phase II and III clinical studies. Br J Dermatol. 2011 Feb 17. doi: 10.1111/j.1365-2133.2011.10257.x.

Accordingly the present invention relates to an in vitro method for predicting the risk of a cardiovascular event in a subject, who was administered with a Th17blocker treatment, said method comprising the step of measuring the level of IL-17 in a blood sample obtained from said subject. Said method may further comprise a step of measuring the level of sVCAM in the blood sample obtained from the subject

As used herein the term “Th17 blocker” refers to any compound that neutralizes the activity of Th17 lymphocytes. The T-helper 17 (Th17) lineage is a subset of memory T cells that is characterized by its CD4(+) status and its ability to make a constellation of cytokines including interleukin-17A (IL-17A), IL-17F and IL-22. Th17 blockers are particularly useful for the treatment of inflammatory diseases and autoimmune diseases. Said diseases include but are not limited to psoriasis, inflammatory bowel diseases, rheumatoid arthritis, inflammatory dermatoses and multiple sclerosis.

The signature cytokine of Th17 cells is the cytokine IL-17A and accordingly the Th17 blocker may consist in an anti-IL-17 antagonist. The IL-17 antagonist may inhibit the expression of IL-17or IL-17RorIL-1 7RC or may inhibit IL-7 signaling by directly or indirectly interacting with one or more of these polypeptides to prevent a functional ligand-receptor interaction. In some preferred embodiments, the IL-17 antagonist is an antibody or antibody fragment that binds to and inhibits the activity of either IL-17, IL17R or IL17C. In one particularly preferred embodiment, the IL-17 antagonist is a monoclonal antibody that specifically binds to IL-17. In other preferred embodiments, the IL-17 antagonist is a bispecific antibody that binds to and inhibits the activity of IL-23p19 and IL-17; IL-23p19 and IL-17RA; IL-23R and IL-17; or IL-23R and IL-17RA. In another particularly preferred embodiment, the IL-17 antagonist is a bispecific antibody that binds to and inhibits the activity of IL-23p19 and IL-17. Examples of anti-IL17 antibodies include but are not limited to LY2439821, AIN457, and AMG827.

Another cytokine intimately associated with TH17 cells is IL-23. This cytokine consists of two proteins, the p19 subunit and the shared IL-12p40 subunit and was shown to drive the differentiation of naive T cells into TH17 cells. Accordingly, Th17 blocker may consist in an IL-23 antagonist. Typically, the IL-23 antagonist may inhibit the expression of either subunit of the cytokine (IL-23p19 or p40), either subunit of the functional receptor (IL-23R or IL-12betal), or may inhibit IL-23 signaling by directly or indirectly interacting with one or more of these polypeptides to prevent a functional ligand-receptor interaction. In some preferred embodiments, the IL-23 antagonist is an antibody or antibody fragment that binds to and inhibits the activity of either IL-23p 19 or IL-23R. In one embodiment, the IL-23 antagonist is a monoclonal antibody that specifically binds to IL-23p19. In one embodiment, the IL-23 antagonist is a monoclonal antibody that specifically binds to IL-12p40. Examples of anti-IL23 antibodies include but are not limited to ustekinumab and Briakinumab.

Kits of the Invention A further object of the invention relates to a kit for performing the above described method, said kit comprising means for measuring the level of IL-17 and optionally means for measuring level of sVCAM-1 in the blood sample obtained from the subject. In a particular embodiment, said means for measuring the level of IL-17 is an antibody that interacts specifically with IL-17. In another embodiment, said means for measuring the level of IL-17 may be an aptamer or any other binding partner that specifically recognizes IL-17.

In another embodiment, said kit further comprises means for measuring the level of sVCAM-1. In a particular embodiment, said means for measuring the level of sVCAM-1 is an antibody that interacts specifically with sVCAM-1. In another embodiment, said means for measuring the level of sVCAM-1 may be an aptamer or any other binding partner that specifically recognizes sVCAM-1.

In another embodiment, the kit of the invention may further comprise means for measuring at least one cardiovascular risk factor selected in the group of Framingham Risk Score (FRS), CRP, IgM ICof apoB100 or IgM MDA-LDL, Lp-PLA2, sPLA2 activity and sPLA2 mass.

Said binding partner(s) can be tagged for an easier detection. It may or may not be immobilized on a substrate surface (e.g., beads, array, and the like). For example, an inventive kit may include an array for predicting the risk of having a cardiovascular event as provided herein. Alternatively, a substrate surface (e.g. membrane) may be included in an inventive kit for immobilization of the binding partner (e.g., via gel electrophoresis and transfer to membrane).

In addition, a kit of the invention generally also comprises at least one reagent for the detection of a complex between binding partner included in the kit and biomarker of the invention.

Depending on the procedure, the kit may further comprise one or more of: extraction buffer and/or reagents, western blotting buffer and/or reagents, and detection means. Protocols for using these buffers and reagents for performing different steps of the procedure may be included in the kit.

The different reagents included in a kit of the invention may be supplied in a solid (e.g. lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit comprises instructions for using its components for the prediction of a cardiovascular event in a subject according to a method of the invention. Instructions for using the kit according to methods of the invention may comprise instructions for processing the biological sample obtained from the subject and/or for performing the test, or instructions for interpreting the results. A kit may also contain a notice in the form prescribed by a governmental agency regulating the manufacture, use or sale of pharmaceuticals or biological products.

The invention will be further illustrated by the following figures and examples. However, these examples and figures should not be interpreted in any way as limiting the scope of the present invention.

### EXAMPLE

### Methods

### Study Population

The population and methods of the French registry of Acute ST-elevation and non-ST-elevation Myocardial Infarction (FAST-MI) have been described in detail in previous publications (Cambou J P, Simon T, Mulak G, Bataille V, Danchin N. The french registry of acute st elevation or non-st-elevation myocardial infarction (fast-mi): Study design and baseline characteristics. Arch Mal Coeur Vaiss. 2007;100:524-534. ; Simon T, Verstuyft C, Mary-Krause M, et al. Genetic determinants of response to clopidogrel and cardiovascular events. N Engl J Med. 2009;360:363-375.). Briefly, all subjects ≧18 years of age were included in the registry if they had elevated serum markers of myocardial necrosis higher than twice the upper limit of normal for creatine kinase, creatine kinase-MB or elevated troponins, and either symptoms compatible with acute MI and/or electrocardiographic changes on at least two contiguous leads with pathologic Q waves (≧0.04 sec) and/or persisting ST elevation or depression >0.1 mV. The time from symptom onset to intensive care unit admission had to be <48 h. Subjects were managed according to usual practice; treatment was not affected by participation in the registry. Of the 374 centres in France that treated subjects with acute MI at that time, 223 (60%) participated in the registry. Among these, 100 centres recruited 1029 subjects who to a serum bank. Written informed consent was provided by each subject.

Their baseline characteristics were comparable to the overall population of the registry. More than 99% of subjects were Caucasians. Follow-up was collected through contacts with the subjects' physicians, the subjects themselves or their family, and registry offices of their birthplace. One-year follow-up was >99% complete. The study was reviewed by the Committee for the Protection of Human Subjects in Biomedical Research of Saint Antoine University Hospital and the data file was declared to the Commission Nationale Informatique et Liberte.

### Blood Sampling and Measurements

Blood samples used for this study were recovered at the time of admission to the intensive care unit (<48 h from symptom onset). Blood samples were stored at −80° C. at the Department of Clinical Pharmacology, University of Pierre et Marie Curie. All samples were identified by number only and were analysed in random order. Serum concentrations of IL-17 were measured using the flow cytomix assay (Bender Med Systems) with a detection limit at 2.5 pg/ml. Soluble VCAM-1 was measured using an ELISA assay (Bender Med Systems). The limit of detection for sVCAM-1 was 0.6 ng/ml. Among the 1029 subjects who contributed to a serum bank, results for IL-17 and sVCAM-1 levels were obtained for 981 subjects and 966 respectively (missing measures or hemolysis).

### Cell Adhesion Assay

Human peripheral blood mononuclear cells (PBMC) were isolated on a PANCOLL gradient (Biotech GmbH). After a washout with PBS, cells were stained with the use of a fluorescent probe (0.5 μM ; CellTracker Orange CMTMR; Molecular Probes). Briefly, the cells were incubated with the fluorescent probe during 30 minutes in RPMI and resuspended in culture medium after a 30 minutes washout for adhesion assay. Human Umbilical Cord Endothelial Cells (HUVEC) (Pomocell) were plated in 48-well plates and stimulated for 24 hours with 10 ng/ml TNF-α (R&D Systems) in the absence or in the presence of recombinant IL-17 (R&D Systems) at 10 ng/ml or 100 ng/ml prior to cell adhesion assay. After washout, fluorescent PBMC cells were made to adhere to HUVEC for one hour. After two more washouts, adherent cells were fixed in 4% paraformaldehyde and counted in 5 different fields per condition under fluorescence microscope (Zeiss microscope).

### ELISA Assay

HUVEC were stimulated with 100 ng/ml of TNF-α in presence or not of either 10 ng/ml or 100 ng/ml of recombinant IL-17 during 48 hr. Then supernatants were collected for ELISA assay of sVCAM-1 (Bender Med Systems) and IL-6 (BD Biosciences). The limit of detection for VCAM-1 was 0.6 ng/ml, and the lowest concentration of standard sample for IL-6 was 4.7 pg/ml.

### Statistical Analysis

An outcome event was defined as all-cause death or non-fatal MI during the one-year follow-up period. The primary endpoint was defined as a composite of all-cause death and non-fatal MI, and was adjudicated by a committee whose members were unaware of subjects' medications, and blood measurements. Continuous variables are described as mean SD and categorical variables as frequencies and percentages. Serum levels of IL-17, CRP, and sVCAM were log-transformed to remove positive skewness, before being used as continuous variables. Baseline demographic and clinical characteristics, treatment factors, and therapeutic management during hospitalisation were compared among the median range of IL-17 levels using chi-square or Fisher' s exact tests for discrete variables, and by unpaired T tests, Wilcoxon sign-rank tests for continuous variables. Median level IL-17 was based on the distribution among subjects without events during follow-up. Survival curves according to median 1L17 level are estimated using the Kaplan Meier estimator. We used a multivariable Cox proportional-hazards model to assess the independent prognostic value of variables with the primary endpoint during the 1-year follow-up period. The multivariable model comprised sex, age, previous or current smoking, family history of coronary disease, history of hypertension, acute MI, heart failure, renal failure, diabetes, heart rate at admission, Killip class, left ventricular ejection fraction, hospital management (including reperfusion therapy, statins, betablockers, clopidogrel, diuretics, digitalis, heparin), and log CRP levels. Results are expressed as hazard ratios for Cox models with 95% confidence intervals (CIs). All statistical tests were two-sided and performed using SAS software version 9.1. For analysis of the cell adhesion and in vitro cytokines assays, we performed multiple comparisons using ANOVA and Bonferroni/Dunn test.

### Results

Baseline demographics and clinical presentation Of the 981 subjects enrolled, 128 subjects (15%) died or had a myocardial infarction during the one-year follow-up period. Subjects who died or had a myocardial infarction during follow-up were older (75 vs 65 years) with a higher proportion of females (42% vs 28%), than those without an outcome event. They also had a higher rate of hypertension (79% vs 59%), diabetes (52% vs 29%), prior heart failure (15% vs 3%), prior myocardial infarction (27% vs 16%), prior stroke or transient ischemic event (14% vs 7%), and chronic renal failure (14% vs 4%) (all P<0.001). They were less likely to be on statin therapy (67% vs 80%), betablockers (49% vs 74%), clopidogrel, heparin, but more likely to be on diuretics, or digoxin (all P<0.002) compared with subjects without outcome event during followup. Subjects who had an event were at higher risk of hospital death according to the GRACE (Global Registry of Acute Coronary Events) risk score (185 vs 159) and a fewer percentage had undergone coronary angioplasty PCI (44% vs 71%) or thrombolysis (8% vs 18%) during hospitalization (all P<0.001). However as shown in Table 1, the baseline characteristics of subjects according to median of IL-17 levels did not differ with the exception of a higher rate of previous or current smokers in those with baseline IL-17 levels <6.84 pg/ml (58% vs 51% in subjects with baseline IL-17 >6.84 pg/ml, P=0.03). No significant correlation was observed between IL-17 and CRP (Pearson Coefficient=-2%, P=0.47). A weak positive correlation of IL-17 was observed with VCAM-1 (Pearson Coefficient=10%, P=0.001).

### IL-17 and Clinical Outcomes at One Year

At one year, the events rate for death and MI was higher in subjects with IL-17 levels below 6.84 pg/ml (15%) compared to those with levels above the median (10%). The corresponding hazard ratio for events rate was 1.53 (95% CI=1.07-2.18) (P=0.02). After adjustment for known cardiovascular risk factors, CRP, and treatments including statins, low IL-17 levels remained an independent correlate of the risk of death or MI, HR 1.51 (1.05-2.17) (P=0.028). We also tested for trend over tertiles of IL-17 to examine the association over a wider range of IL-17 levels. Compared to tertile 1, chosen as reference, adjusted HRs were 0.65 (0.42-0.99) and 0.63 (0.41-0.97) for tertile 2 and tertile 3, respectively. The Cochran-Armitage trend test was significant (P=0.021).

### Soluble VCAM-1 and Clinical Outcomes at One Year

At one year, 18% of subjects among those with sVCAM-1 levels above the median (≧678.49 ng/mL) died or had a non-fatal MI compared to 6.4% in those with levels below the median (<678.49 ng/mL). sVCAM-1 levels above the median were associated with an increased risk of death or recurrent MI at one year (HR=2.97, 95% CI=1.96-4.51 compared to sVCAM-1 below the median, P<0.0001). After adjustment for known cardiovascular risk factors, CRP, IL-17 levels, and treatments including statins, high sVCAM-1 levels remained an independent correlate of outcome events (HR=1.82, 95% CI=1.17-2.84, P=0.009).

### Combined Assessment of IL-17 and Soluble VCAM-1 to Predict Clinical Outcomes

At one year, 46% of death and MI occurred in subjects with low (below the median) baseline IL-17 and high (above the median) baseline sVCAM-1 levels compared to 9% in those subjects with high baseline IL-17 and low baseline sVCAM-1. The corresponding hazard ratio for events rate was 4.49 (95% CI=2.35-8.57) (P<0.0001) and remained at a particularly high risk of death and recurrent MI in the cox multivariate analysis (adjusted HR=2.56, CI 1.30-5.04, P=0.006). There was no significant interaction between IL-17 and VCAM-1 (P=0.63).

IL-17 Reduces Adherence of Mononuclear Cells and Endothelial sVCAM-1 Production

In light of these results and our previous finding that IL-17 protective role in a mouse model of atherosclerosis was associated with reduced endothelial VCAM-1 expression, we tested the hypothesis that IL-17 modulates human mononuclear cell adhesion on endothelial cells. We observed a significant reduction of PBMC adhesion to TNFα-activated HUVEC in the presence of IL-17, which was associated with a significant reduction of sVCAM-1 expression. As a control, IL-17 enhanced IL-6 production in the same supernatants. These results support an important role of IL-17 in the regulation of mononuclear cell recruitment and vascular inflammation.

### Discussion:

The major finding of this study is that elevated levels of IL-17 are associated with a better outcome in subjects with acute MI, supporting a protective regulatory role of IL-17 in coronary heart disease. Moreover, the highest risk of death and recurrent MI was observed in subjects with low levels of IL-17 and high levels of sVCAM-1, suggesting an important modulatory role of IL-17 on vascular inflammation.

Only few studies have previously reported measurements of circulating IL-17 levels in subjects with coronary heart disease. The first published investigations, performed in less than 26 Chinese subjects per group, suggested increased levels of IL-17 and Th17 subset in subjects with coronary artery disease, more particularly in subjects with acute coronary syndromes (Hashmi S, Zeng Q T. Role of interleukin-17 and interleukin-17-induced cytokines interleukin-6 and interleukin-8 in unstable coronary artery disease. Coron Artery Dis. 2006;17:699-706; Cheng X, Yu X, Ding Y J, et al. The th17/treg imbalance in subjects with acute coronary syndrome. Clin Immunol. 2008;127:89-97.). However, these results were not replicated in Caucasians (Patel K D, Murphy R T, White M, et al. Interleukin 17: An unlikely marker of acute coronary syndrome? Atherosclerosis. 2009;205:33-34; Eid RE, Rao DA, Zhou J, et al. Interleukin-17 and interferon-{gamma} are produced concomitantly by human coronary artery-infiltrating t cells and act synergistically on vascular smooth muscle cells. Circulation. 2009;119:1424-32.). Eid et al. found no difference in IL-17 levels between subjects with coronary artery disease (n=108) and referent outsubjects without a diagnosis of coronary atherosclerosis (n=59) (Eid R E, Rao D A, Zhou J, et al. Interleukin-17 and interferon-{gamma} are produced concomitantly by human coronary artery-infiltrating t cells and act synergistically on vascular smooth muscle cells. Circulation. 2009;119:1424-32.). In addition, IL-17 levels did not differ between subjects with stable and those with unstable coronary syndromes (Eid R E, Rao D A, Zhou J, et al. Interleukin-17 and interferon-{gamma} are produced concomitantly by human coronary artery-infiltrating t cells and act synergistically on vascular smooth muscle cells. Circulation. 2009;119:1424-32.). Importantly, the previous clinical studies have generated assumption about the role of IL-17 in coronary artery disease but none had assessed the relationship between IL-17 levels and cardiovascular outcomes. Our study is the first to tackle this issue and shows that the detection of elevated levels of IL-17 in subjects with acute MI is associated with a better cardiovascular outcome, i.e., reduced mortality and recurrent MI after one year of follow-up.

Thus, the currently held dogma that IL-17 promotes coronary artery disease requires reconsideration.

### REFERENCES

Throughout this application, various references describe the state of the art to which this invention pertains. The disclosures of these references are hereby incorporated by reference into the present disclosure.

1. An in vitro method for predicting the risk of a cardiovascular event in a subject, said method comprising the steps of
i) measuring a level of IL-17 in a blood sample obtained from said subject by
contacting the blood sample with a binding partner that is specific for the IL-17, wherein the step of contacting forms an IL-17-binding partner complex, and
detecting the IL-17-binding partner complex as a measure of the level of IL-17; and
ii) comparing the level measured at step i) with a reference value, wherein a difference between said IL-17 level and said reference value is indicative of a risk of having a cardiovascular event.

2. The in vitro method according to claim 1 which further comprises a step of measuring a level of soluble vascular cell adhesion molecule-1 (sVCAM-1) in the blood sample obtained from the subject.

3. The in vitro method according to claim 1 wherein a Th17 blocker treatment was administered to said subject.

4. A kit for performing the method according to claim 2 which comprises means for measuring the level of IL-17 and means for measuring the levels of sVCAM-1 in a blood sample.

5. The method of claim 1, wherein the binding partner is an antibody or an aptamer.

6. A method for treating a subject at risk of a cardiovascular event, comprising the steps of:
analyzing a blood sample obtained from said subject by
contacting the blood sample with a binding partner that is specific for the IL-17, wherein the step of contacting forms an IL-17-binding partner complex,
detecting the IL-17-binding partner complex as a measure of a level of IL-17; and
comparing the level measured in said detecting step with a reference value, and
when a difference between said IL-17 level and said reference value is indicative of said risk of having said cardiovascular event, selecting a treatment regimen for said subject.

7. The method of claim 6 wherein said treatment regimen selected in said selecting step avoids the use of inhibitors of the IL-17 pathway.

8. The method of claim 6 wherein said subject is administered a TH17 blocker prior to said analyzing and selecting steps.

9. The method of claim 8, wherein said TH17 blocker is selected from the group consisting of an anti IL 17 antagonist and an IL 23 antagonist.

10. A method for monitoring effectiveness of a treatment for a subject at risk of a cardiovascular event, comprising the steps of:
analyzing blood samples obtained from said subject before and after receiving said treatment by
contacting the blood sample with a binding partner that is specific for the IL-17, wherein the step of contacting forms an IL-17-binding partner complex,
detecting the IL-17-binding partner complex as a measure of a level of IL-17; and
comparing the level measured in said detecting step with a reference value, and
determining effectiveness of said treatment based on differences between a comparison made for a first blood sample before said treatment and a comparison made for a second blood sample after said treatment.

11. The method of claim 10 wherein said subject is administered a TH17 blocker prior to said analyzing and selecting steps.

12. The method of claim 8, wherein said TH17 blocker is selected from the group consisting of an anti IL 17 antagonist and an IL 23 antagonist.
